(19) United States
(12) Patent Application Publication
(10) Pub. No.: US 2016/0223522 A1
(43) Pub. Date: Aug. 4, 2016

(54) ASSAY FOR DETERMINING RELATIVE REDOX CHANGES IN LIVING CELLS AND ASSOCIATED DEVICES, SYSTEMS, AND METHODS

(71) Applicant: University of Utah Research Foundation, Salt Lake City, UT (US)

(72) Inventors: Shayne Squires, Coalville, UT (US); Ivor Benjamin, Salt Lake City, UT (US)

(73) Assignee: University of Utah Research Foundation, Salt Lake City, UT (US)

(21) Appl. No.: 14/997,332

(22) Filed: Jan. 15, 2016

Related U.S. Application Data
(63) Continuation-in-part of application No. 14/252,522, filed on Apr. 14, 2014.
(60) Provisional application No. 61/811,530, filed on Apr. 12, 2013.

(51) Int. Cl. G01N 33/50; G01N 21/64; C07K 7/08; G01N 33/58

(52) U.S. Cl. CPC G01N 33/5008; G01N 33/582; G01N 21/6428; C07K 7/08; G01N 2021/6432

(57) ABSTRACT

The present disclosure provides conjugates, systems, devices, and methods for detecting cellular redox state. In one aspect, for example, a conjugate for detecting cellular redox state can include a first segment including a cell penetrating peptide conjugated to a first detection molecule, and a second segment including a cargo peptide conjugated to a second detection molecule, wherein the first segment and the second segment are coupled together by a redox-sensitive linkage, and wherein the first detection molecule and the second detection molecule have properties that allow linked proximity detection. In one specific example, the first detection molecule and the second detection molecule include a fluorophore/quencher pair.

### PRIORITY DATA

This application is a continuation-in-part of U.S. application Ser. No. 14/252,522, filed on Apr. 14, 2014, which claims the benefit of U.S. provisional patent application Ser. No. 61/811,530, filed on Apr. 12, 2013, each of which is incorporated herein by reference.

### BACKGROUND OF THE INVENTION

Cell penetrating peptides (CPP) exhibit unique properties for translocation across cellular membranes and non-endocytic uptake into mammalian cells. Model Amphipathic Peptide (MAP), for example, has amino acid sequence KLALKLALKALKAALKLA-NH₂ (SEQ ID 003) and is thought to adopt an alpha-helical conformation where hydrophobic side chains align along one hemicircumference of the α-helix and positively charged side chains align along the opposite hemicircumference. In the course of studying MAP's interaction with the plasma membrane, the cell-penetrating property was discovered that paved the way for research into the mechanism that govern peptide translocation into mammalian cells.

The mechanisms of cell peptide internalization and localization remain under active investigation. A cellular penetration mechanism was originally inferred to be nonendocytic based upon observed uptake at 0° C. and following energy depletion. However, in subsequent experiments, various maneuvers commonly believed to inhibit endocytosis yielded mixed results with evidence for and against endocytic uptake. Peptide uptake was decreased but not abolished after treatment of the cells with 2-deoxyglucose, motivating the inference that uptake is mediated by both energy-dependent and -independent mechanisms. Of the labeled cell-associated peptide, 50% was membrane bound, 30% was inserted into the membrane, and 20% was fully internalized. Using giant lipid membrane vesicles with a lipid bilayer content similar to intact cells but without the ability to endocytose, it was demonstrated that MAP uptake persists even without endocytosis. Upon internalization, the subcellular distribution of MAP has been reported to include both cytosolic and nuclear compartments.

### BRIEF DESCRIPTION OF THE DRAWINGS

For a fuller understanding of the nature and advantage of the present disclosure, reference is being made to the following detailed description of various embodiments and in connection with the accompanying drawings, in which:

FIG. 1a shows a graphical representation of data according to one embodiment of the present disclosure.

FIG. 1b shows a graphical representation of data according to one embodiment of the present disclosure.

FIG. 1c shows a graphical representation of data according to one embodiment of the present disclosure.

FIG. 1d shows a graphical representation of data according to one embodiment of the present disclosure.

FIG. 2a shows image data according to one embodiment of the present disclosure.

FIG. 2b shows image data according to one embodiment of the present disclosure.

FIG. 2c shows image data according to one embodiment of the present disclosure.

FIG. 2d shows image data according to one embodiment of the present disclosure.

FIG. 3a shows image data according to one embodiment of the present disclosure.

FIG. 3b shows image data according to one embodiment of the present disclosure.

FIG. 3c shows image data according to one embodiment of the present disclosure.

FIG. 3d shows a graphical representation of data according to one embodiment of the present disclosure.

FIG. 3e shows a graphical representation of data according to one embodiment of the present disclosure.

FIG. 3f shows a graphical representation of data according to one embodiment of the present disclosure.

FIG. 3g shows a graphical representation of data according to one embodiment of the present disclosure.

FIG. 3h shows a graphical representation of data according to one embodiment of the present disclosure.

FIG. 3i shows a graphical representation of data according to one embodiment of the present disclosure.

FIG. 4a shows a graphical representation of data according to one embodiment of the present disclosure.

FIG. 4b shows a graphical representation of data according to one embodiment of the present disclosure.

FIG. 4c shows a graphical representation of data according to one embodiment of the present disclosure.

FIG. 4d shows a graphical representation of data according to one embodiment of the present disclosure.

FIG. 5 shows a graphical representation of data according to one embodiment of the present disclosure.

FIG. 6a shows a graphical representation of data according to one embodiment of the present disclosure.

FIG. 6b shows a graphical representation of data according to one embodiment of the present disclosure.

FIG. 7 shows a graphical representation of data according to one embodiment of the present disclosure.

FIG. 8a shows a graphical representation of data according to one embodiment of the present disclosure.

FIG. 8b shows a graphical representation of data according to one embodiment of the present disclosure.

FIG. 9a shows a graphical representation of data according to one embodiment of the present disclosure.

FIG. 9b shows a graphical representation of data according to one embodiment of the present disclosure.

FIG. 9c shows a graphical representation of data according to one embodiment of the present disclosure.

FIG. 10a shows a graphical representation of data according to one embodiment of the present disclosure.

FIG. 10b shows a graphical representation of data according to one embodiment of the present disclosure.

FIG. 10c shows a graphical representation of data according to one embodiment of the present disclosure.

FIG. 11a shows a graphical representation of data according to one embodiment of the present disclosure.

FIG. 11b shows a graphical representation of data according to one embodiment of the present disclosure.

FIG. 11c shows a graphical representation of data according to one embodiment of the present disclosure.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

### DEFINITIONS

In describing and claiming the present invention, the following terminology will be used in accordance with the definitions set forth below.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Disclosure

The present disclosure provides conjugates, systems, devices, and methods for detecting cellular redox state via the cellular delivery of cell penetrating peptides (CPP). In one example embodiment, cellular redox state can be detected via disulfide-linked cargo delivery along a model amphipathic peptide (MAP)-mediated pathway, although other cellular delivery mechanisms and cargo delivery molecules are contemplated, all of which are considered to be within the present scope. Variations in the delivery of disulfide-linked or other cargo moieties can also be used as an effective indicator of relative changes in cellular redox state.

MAP and numerous other cell-penetrating peptides can shuttle proteins, nucleic acids, small polar compounds, and the like, across the plasma membrane. MAP conjugated to polylysine can be used to form multiplexes with siRNA against green fluorescent protein (GFP). The MAP-siRNA multiplexes are more effective at inhibiting GFP expression than Lipofectamine® siRNA transduction. Additionally, MAP can be reversibly conjugated through a disulfide bond, for example, or irreversibly conjugated through a thioether linkage, for example, to peptide nucleic acid (PNA) sequences and tested for cellular uptake in a luciferase expression assay. Both reversible and irreversible linkages result in intracellular delivery of PNA. Interestingly, luciferase expression is enhanced by treatment with chloroquine, suggesting that a significant amount of MAP-conjugated PNA is sequestered in endosomes, and that endosomal release improves nuclear uptake. By comparison, cytochrome c reversibly linked to MAP via a disulfide bond is taken up by HeLa cells and partially transitioned from vesicles to cytosol, enabling apoptosis in response to treatment with MG132. Thioether-linked cytochrome c does not transition to cytosol and does not enhance apoptosis in response to MG132 treatment.

Cell-uptake kinetics of CPP can be assayed using disulfide-linked cargo. For example, a cargo peptide comprising the sequence CLKANL (SEQ ID 001) that is N-terminally labeled with the fluorophore such as, for example, 2-aminobenzoic acid (Abz), can be joined to Cys-3-nitrotyrosine-MAP through a disulfide bond. The 3-nitrotyrosine moiety quenches emission from Abz while the disulfide bond remains intact. Upon entry into the cell, the disulfide bond is reduced, enabling Abz fluorescence and detection of internalization. Among four cell-penetrating peptides (MAP, penetratin, TAT, and transportan), MAP shows the fastest cellular uptake, and its internalized fraction reaches 60% within 30 minutes of incubation.

MAP-mediated cellular delivery of disulfide-linked cargo varies with cellular redox state, and this variation can be used to detect relative changes in cellular redox state. By conjugating MAP to 5(6) carboxytetramethylrhodamine (TAMRA)-cysteine at the N-terminus and the peptide CLKANL (SEQ ID 001) to fluorescein amidite (FAM) at the N-terminus, for example, a disulfide-linked fluorescence resonance energy transfer (FRET) pair is created that is capable of separately interrogating cellular entry and disulfide reduction. This disulfide-linked CPP construct is hereinafter referred to as “reductide.” Cellular internalization of C-MAP can be conveniently tracked by TAMRA fluorescence, for example, which normally quenches FAM fluorescence unless the disulfide is reduced, thus instantaneously enabling this event to be monitored by FAM fluorescence—both in vitro and in vivo. Furthermore, reductide can detect relative changes in cellular redox state in living cells.

In one example embodiment, a conjugate for detecting cellular redox state can include a first segment including a cell penetrating peptide conjugated to a first detection molecule, and a second segment including a cargo peptide conjugated to a second detection molecule, wherein the first segment and the second segment are coupled together by a redox-sensitive linkage. The first detection molecule and the second detection molecule have properties that allow linked proximity detection. Linked proximity detection includes any mechanism that generates a detectable or otherwise measurable proximity signal that is dependent upon the relative proximity of two molecules. In some cases, the proximity signal can be generated when the two molecules are close in proximity, while in other cases the proximity signal can be generated when the two molecules are far apart in proximity. Thus, whether two molecules are linked or unlinked can be determined by detecting or otherwise measuring the presence or absence of the proximity signal. It is noted that any mechanism that can be utilized for detecting the proximity of two molecules is considered to be within the present scope.

In one specific example, the first detection molecule and the second detection molecule include a fluorophore/quencher pair. While any fluorophore/quencher pair that can be appropriately utilized are contemplated, in one non-limiting example the first detection molecule is 5(6)carboxytetramethylrhodamine-cysteine and the second detection molecule is fluorescein amidite. In this case, the 5(6)carboxytetramethylrhodamine-cysteine quenches fluorescence of fluorescein amidite when in linked proximity. Additionally, while other combinations are contemplated, in one example the cell penetrating peptide is conjugated at an N-terminus to the 5(6)carboxytetramethylrhodamine-cysteine and the cargo peptide is conjugated at an N-terminus to the fluorescein amidite.

Furthermore, other non-limiting fluorophore/quencher examples can include fluorescein quenched by rhodamine; dabcyl quenching Oregon Green™ 488-X, 6-FAM, Cy3, TAMRA, or Texas Red; BHQ-1 quenching Oregon Green, 6-FAM, Rhodamine Green, TET, JOE, Cy3, or TAMRA; BHQ-2 quenching HEX, ROX, BODIPY, or Cy5; Iowa Black quenching Cy3, Cy5, or BODIPY; and self-quenching fluorophores such as, for example, near infrared fluorophores that can quench themselves when brought into close proximity with one another. One non-limiting example can include Cy5.5.

Any suitable cell penetrating peptide is considered to be within the present scope. In one example embodiment, however, the cell penetrating peptide can be a cationic cell penetrating peptide. Non-limiting examples of cell penetrating peptides can include Tat-derived cell penetrating peptides, penetratins, transportan and transportan-related peptides, MAPs, and the like, including appropriate combinations thereof. Tat-derived cell-penetrating peptides can be derived from the HIV encoded Tat protein, and typically contain a number of positively charged amino acid residues. Penetratins and transportans can also be characterized by multiple positively charged amino acids. Cell-penetrating peptides with a number of positively charged side chains can potentially be used in the construct to interrogate intracellular redox state.

Furthermore, any linkage capable of allowing redox state to be assayed is considered to be within the present scope. Non-limiting examples can include disulfide linkages, linkages comprising substrates for redox-controlled enzymes, linkages comprising substrates for enzymes that are up regulated or expressed in response to cellular redox changes, and the like, including appropriate combinations thereof. In one specific example, the redox-sensitive linkage can be a disulfide linkage.

Similarly, any useful cargo peptide is considered to be within the present scope. The cargo peptide can be designed to merely be a structural scaffolding for the construct, or the cargo peptide can be designed to have a therapeutic or other use. In one example, the cargo peptide can be from one to fifty amino acids in length. In another example, the cargo peptide can include fluorescent proteins, bioluminescent proteins, and the like, including appropriate combinations thereof. In yet another aspect, the cargo peptide can have the sequence CLKANL (SEQ ID 001).

The present disclosure additionally provides methods of detecting cellular redox state, including introducing the conjugate described according to aspects of the present disclosure into a cell and measuring linked proximity of the first detection molecule and the second detection molecule to detect cleavage of the redox sensitive linkage, thus determining a cellular redox state. In one embodiment, the method can further include detecting at least one of the first or second detection molecules to determine uptake of the conjugate by the cell. For example, in one non-limiting case, the first detection molecule can quench the second detection molecule when the conjugate is linked. Thus, the first detection molecule can be detected to quantify uptake into the cell. Upon cleavage, the signal from the second molecule becomes detectable, thus allowing measurement of redox state. In other cases, the second detection molecule can quench the first detection molecule when the conjugate is linked, and it is the second detection molecule that can be detected in order to quantify uptake and the cell. Upon cleavage, the signal from the first molecule becomes detectable, thus facilitating the measurement of redox state. Furthermore, in many cases the cell can include a population of cells, and thus cellular redox state can be monitored across the population of cells to determine a relative change in cellular redox state.

A variety of potential applications for the present constructs and methods are contemplated, and any such application is considered to be within the present scope. In one aspect, for example, the method/conjugate can be used for discovering redox modifying agents. The conjugate assay can be used to screen a chemical library to find agents that increase, decrease, or otherwise modify intracellular reduction. It can also be used to screen biological agents for the same or similar embodiment effects.

In one example, the method/conjugate can be used for the discovery of cardiac antiarrhythmic agents, anticonvulsant agents to treat epilepsy for example, and the like. The conjugate assay can thus be used to screen a chemical library to find agents that increase the trans-plasma membrane electrical potential difference, or in other words induce the intracellular space to have a more negative charge relative to the extracellular space. Such agents would result in cellular hyperpolarization, thus decreasing the likelihood of cell depolarization.

In another example, the method/conjugate can be used for optical imaging in a subject to detect tissue redox changes in vivo. A positively charged cell-penetrating peptide such as MAP, for example, can be conjugated to a near-infrared probe and cysteine. The resulting conjugate can be dimerized through a disulfide bond and administered to a subject, by any administration pathway such as intravenously, subcutaneously, and the like. The administered agent concentrates in tissue in proportion to redox state, i.e. more reduced tissue would take up a larger fraction of the administered agent. Through an optical imaging modality, the bio can distribution of the agent could be detected, yielding an image map of redox state in the subject.

In a further example, the method/conjugate can be used for scintigraphic imaging in a subject to detect redox state. Nanoparticles such as gold colloid or HPMA can be conjugated to a positively charged CPP, such as MAP, and labeled with a radionuclide such as 99mTc, 111-In, 123-I, 124-I, or other radionuclide suitable for scintigraphic imaging. The resulting agent can be administered intravenously to a subject and concentrated in tissue in proportion to redox state. A scintigraphic imaging modality can then be used to create an image of the subject representing tissue redox state.

In yet a further aspect, the method/conjugate can be used for magnetic resonance imaging (MRI) in a subject to detect redox state. Nanoparticles such as gold colloid or HPMA can be conjugated to a positively charged cell-penetrating peptide, such as MAP, and labeled with gadolinium or other agent suitable for MRI. The resulting construct can be administered intravenously to a subject and concentrate in tissue in proportion to redox state. MRI can then be used to create an image of the subject representing tissue redox state.

Other non-limiting examples can include an assay to detect relative changes in cellular redox state in vitro, and an assay to detect relative changes in cellular polarity, or in other words plasma membrane electric potential, in vitro.

The present disclosure additionally provides a kit for detecting cellular redox state. Such a kit can include a housing containing a conjugate according to aspects of the present disclosure in a biologically suitable carrier, at least one reagent for use with the conjugate in detecting cellular redox state, and instruction materials describing utilization of the conjugate and the at least one reagent to detect the cellular redox state.

In one non-limiting example, it was sought to determine whether MAP-mediated cellular delivery of disulfide-linked cargo varies with cellular redox state and whether this variation can be used to detect relative changes in cellular redox state. It was found that by conjugating MAP to 5(6) carboxytetramethylrhodamine (TAMRA)-cysteine at the N-terminus and the peptide CLKANL (SEQ ID 001) to fluorescein amidite (FAM) at the N-terminus, a disulfide-linked fluorescence resonance energy transfer (FRET) pair capable of separately interrogating cellular entry and disulfide reduction was created. As described above, this novel disulfide-linked CPP construct is referred to as “reductide.” Cellular internalization of C-MAP can be conveniently tracked by TAMRA fluorescence, which normally quenches FAM fluorescence unless the disulfide is reduced, instantaneously enabling this event to be monitored by FAM fluorescence—both in vitro and in vivo.

### Materials and Methods

### Reagents

N-acetylcysteine (NAC), 1-chloro-2,4-dinitrobenzene (CDNB), reduced glutathione (GSH), and oxidized glutathione (GSSG) were purchased from Sigma-Aldrich (St. Louis, Mo., USA). Dulbecco's Modified Eagle Medium™ (DMEM) and fetal bovine serum (FBS) were obtained from Invitrogen (Grand Island, N.Y., USA). Puromycin was purchased from InvivoGen (San Diego, Calif.). Plasmids (pLPCX) containing the gene for glutaredoxin-1 (Grx1) conjugated via a short linker sequence to cytosolic redox sensitive green fluorescent protein (Grx1-roGFP) were a gift from Dr. Tobias Dick (German Cancer Research Center {DKFZ}, Heidelberg, Germany). The Screen-Well™ REDOX library of 84 redox modifying drugs was obtained from Enzo Life Sciences (Farmingdale, N.Y.).

### Peptide Synthesis and Labeling

Reductide was synthesized using standard FMOC solid phase chemistry as two peptide moieties: cysteine conjugated MAP (Cys-Lys-Leu-Ala-Leu-Lys-Leu-Ala-Leu-Lys-Ala-Leu-Lys-Ala-Ala-Leu-Lys-Leu-Ala-amide) (SEQ ID 002) was conjugated to 5(6) carboxytetramethylrhodamine (TAMRA) through the N-terminus, and the non-cell penetrating peptide with the sequence Cys-Leu-Lys-Ala-Asn-Leu (SEQ ID 001) was conjugated to fluorescein amidite (FAM) through the N-terminus. These two sequences were joined through a disulfide bond. Peptides were purified by HPLC and analyzed by mass spectrometry.

### Cells, Cultures, and Transfections

BJ and IMR90 human fibroblasts and H9c2 rat neonatal cardiomyocytes (ATCC, Manassas, Va., USA) were grown on 10 cm dishes, 6-well plates, or in a 96-well plate in DMEM plus 10% (v/v) FBS and supplemented with 2 mM minimum essential amino acids (Invitrogen). Plasmids containing Grx1-roGFP under CMV promoter control with puromycin resistance genes were transfected into PLAT-E cells using Lipofectamine 2000. Retroviruses were harvested from the PLAT-E cell media at 24 and 48 hours and used to infect H9c2 cells, which were cultivated in the presence of puromycin (up to 4 μg/ml) for 15 population doublings. Stable expression was confirmed by fluorescence microscopy and western blot.

### Reductide Assay in GSH Containing Buffer

Reductide was dissolved in 3% acetic acid to a concentration of 100 μM and immediately diluted 1:100 in tris-buffered saline (TB S) pH 7.4 containing reduced glutathione (GSH) plus or minus oxidized glutathione (GSSG) at the indicated concentrations. It was confirmed that the pH of GSH-containing TBS remained unchanged at 7.4 following dilution of reductide. Reductide-containing GSH buffer was aliquoted into a 96-well plate with black sides and clear bottoms (Costar, Corning, N.Y.). Reductide signal was read in a Synergy HT plate reader (BioTek, Winooski, Vt., USA) at the indicated time points following addition to GSH-containing buffer using excitation and emission wavelengths of 485 nm/528 nm (FAM) or 530 nm/590 nm (TAMRA).

### Fluorescence Microscopy

BJ fibroblasts in normal media were seeded onto 4-chamber glass cover slides (Lab-Tek, Rochester, N.Y., USA) at a density of 30,000 cells per chamber and allowed to attach overnight. For experiments involving redox modifying agents, cells were incubated in a humidified chamber at 37° C. in 5% CO2 in normal media supplemented with NAC 4 mM or CDNB 25 μM for 30 minutes just prior to microscopy. Media was then replaced with normal media supplemented with DAPI for 5 minutes. Media was then exchanged for normal media to which reductide was added to a concentration of 5 μM Live cell imaging was performed using an Olympus FV1000 with cells in a stage incubator at 37° C. in 5% CO₂. Each image was acquired for 200 ms, and repeat imaging was performed for 4.5 hours.

Comparison Between Reductide Signal and roGFP

H9c2 cells stably expressing cytosolic roGFP were seeded into a 96-well plate with black sides at a density of 8,000 cells per well in normal media and allowed to attach overnight. Cells were treated with n-acetylcysteine (NAC) or hydrogen peroxide (H₂O₂) at the indicated concentrations for 60 minutes. Cells were washed once with PBS followed by replacement with normal media. High throughput microscopy was performed using a BD Pathway High Content Bioimager 855. During imaging, cells were maintained at 37° C. in a humidified chamber at 5% CO₂. Images of each well were obtained following laser stimulation at 405 nm and 488 nm. Ratiometric images were constructed using ImageJ (National Institutes of Health, Bethesda, Md.) by dividing pixel by pixel the intensity following stimulation at 405 nm by the intensity following stimulation at 488 nm, following background correction for each. Immediately after imaging cells in a 96-well plate using the Pathway Bioimager as above, media was exchanged with normal media containing reductide 1 μM and incubated for 30 minutes at 37° C. in 5% CO₂ in a humidified chamber. Cells were then assayed on a fluorescence plate reader (Synergy HT; BioTek, Winooski, Vt., USA).

### Reductide Plate Reader Assay in Cells

BJ fibroblasts were trypsinized and re-plated in a 96-well plate (5,000 cells/well) in normal growth medium and allowed to attach overnight. Media was replaced with normal media supplemented with the chemical redox-modifying agent indicated in the “Results” section for the indicated duration of treatment. Cells were subsequently washed one time with PBS and media was replaced with normal media supplemented with reductide 1 μM. Cells were incubated at 37° C. in 5% CO₂ for the indicated time points followed by detection of reductide signal in a plate reader using excitation and emission wavelengths of 485 nm/528 nm.

In order to test the effect of redox modifying agents on development of fluorescent signal in cells which have already taken up reductide, cells growing in a 96-well plate were first incubated with reductide 1 μM for 60 minutes followed by washing with PBS and treatment with NAC or H₂O₂ for 60 minutes. Cells were then assayed in a fluorescent plate reader.

For comparison with monochlorobimane, IMR90 fibroblasts were seeded into a 96 well plate at a density of 50,000 cells per well and attached overnight. Cells were incubated for 60 minutes in media containing NAC or H₂O₂ at the indicated concentrations. Cells were washed twice in 200 μl of PBS. Monochlorobimane was used to assay reduced GSH content in half the wells using the Glutathione Assay Kit available from Sigma (CS 1020, St. Louis, Mo., USA), following the manufacturer's instructions for use in live cells in a plate reader. At the same time, reductide was dissolved in the same assay buffer used for monochlorobimane treatment and added to half of the wells at a concentration of 1 μM. Fluorimetric readings were performed in a Synergy HT plate reader using excitation and emission wavelengths of 485 nm/528 nm for reductide and 390 nm/478 nm for monochlorobimane. Each cell condition was triplicated, and each experiment was repeated two times. Representative results are shown.

For comparison with Alamar Blue™ (Invitrogen, Carlsbad, Calif.), cells in a 96-well plate were washed with PBS followed by four hours of incubation with Alamar Blue diluted 1:10 in normal media according to the manufacturer's instructions. Alamar Blue fluorescence was assayed in a plate reader using excitation and emission wavelengths 540 nm and 590 nm, respectively.

### Flow Cytometry

IMR90 fibroblasts were seeded onto six 10 cm dishes at a density of 1.8×10⁶ cells and allowed to attach overnight. Three dishes were treated with H₂O₂ 600 μM and another three with NAC 4 mM, each in normal media, for one hour. Cells were washed with PBS followed by incubation with reductide 1 μM in normal media for 3, 15, or 30 minutes. Cells were then washed again with PBS followed by trypsinization and collection in normal media without phenol red. Cell concentration was 10⁶ per ml. DAPI was added at 1:500 dilution and cells were analyzed by flow cytometry for TAMRA and FAM fluorescence.

### Statistical Analysis

Data are presented as mean+/−standard deviation. Statistical comparison of differences between two groups of data was carried out using a Student's t-test. Differences between more than two groups of data were analyzed using one-way analysis of variance (ANOVA). P-values<0.05 were considered statistically significant and P-values<0.01 were considered highly significant.

### Results

### Effects of GSH/GSSG on Reductide Redox-Dependent Fluorescence

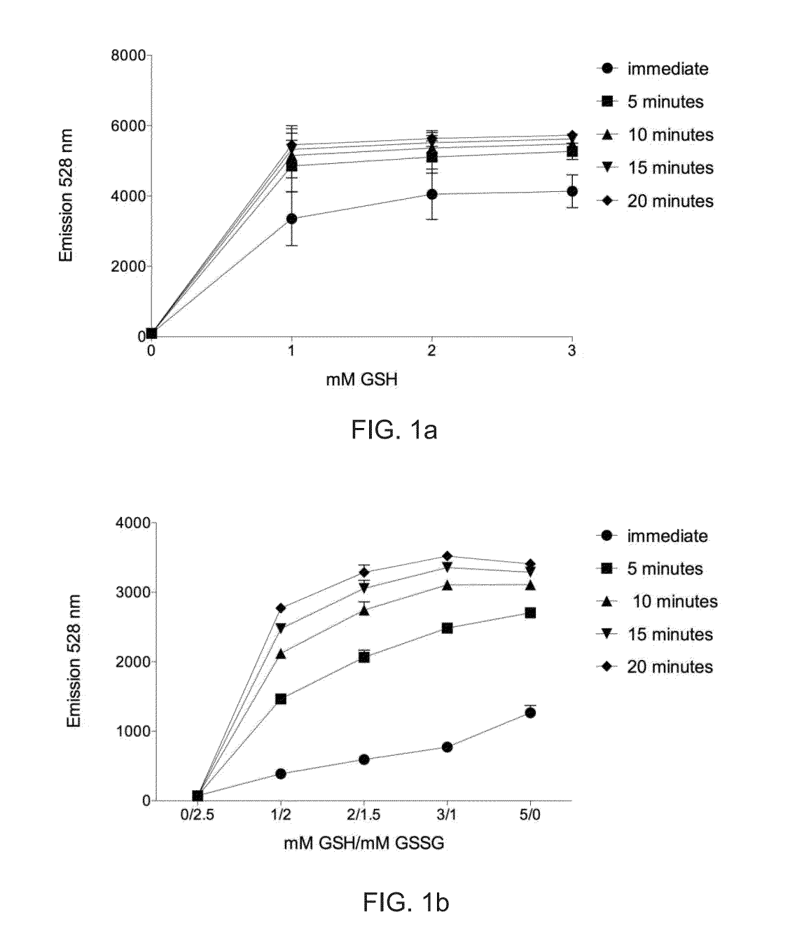

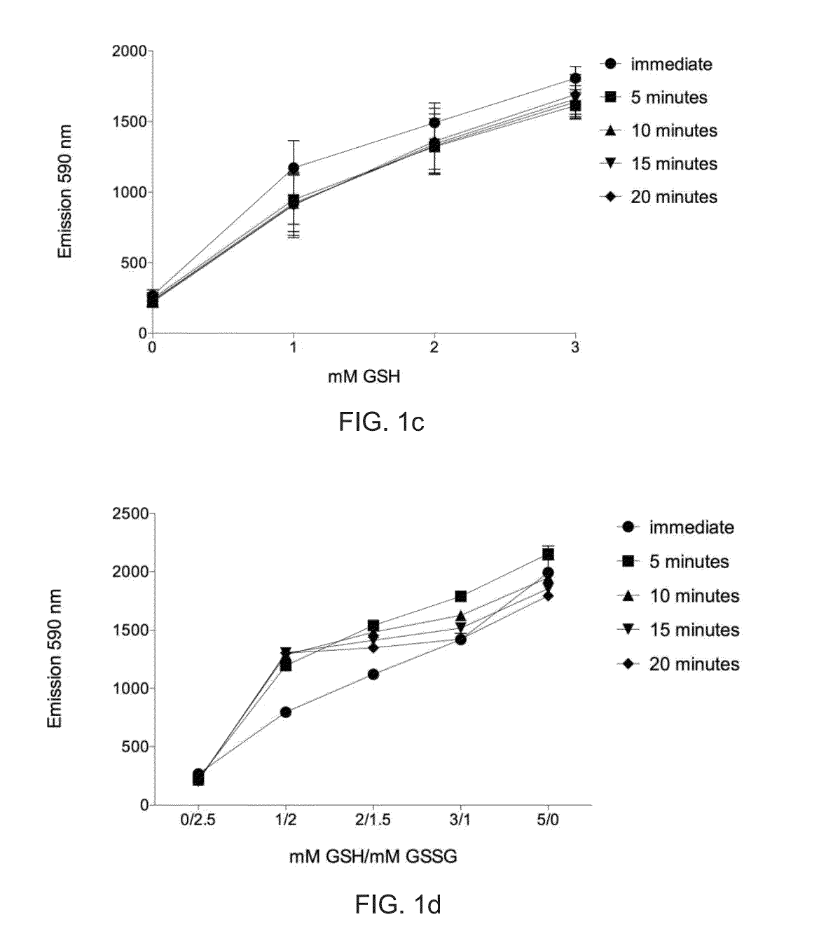

Because the emission signal of FAM is quenched by nearby TAMRA, reduction of the disulfide bond joining the two moieties of reductide triggers separation and achieves readable FAM fluorescence. Reductide was added to buffer containing a GSH pool at least a thousand-fold higher in concentration in order to mimic in vivo peptide reducing conditions. When assayed for fluorescence in a plate reader, in the presence of GSH, stimulation near FAM's absorption maximum (485 nm) resulted in emission at 528 nm. In parallel with increasing GSH concentration, we observed that the emission intensity steadily increased with incubation time, indicating that peptide reduction is a time-dependent process (FIG. 1a).

To assess the effects of GSSG reduction potential on reductide fluorescence, we dissolved reduced and oxidized glutathione (GSH and GSSG) in TBS buffer such that the total glutathione pool was 5 mM (calculated as the concentration of GSH plus twice the concentration of GSSG) and dissolved reductide as before. The presence of added GSSG in the glutathione pool resulted in slower development of reductide fluorescence and a decrease in maximum fluorescence achieved by 20 minutes (FIG. 1b), consistent with the idea that the rate of peptide reduction not only depends on GSH concentration but also on the GSSG reduction potential as calculated using the Nernst equation. In the absence of GSH, there was no increase in emission intensity above initial background levels. In response to either dithiothreitol or NAC, reductide could also be reduced further by other thiol-containing reducing agents (data not shown). TAMRA emission intensity increased with increasing GSH concentration but not in a time-dependent manner, demonstrating lack of reciprocity with FAM's time-dependent increase in emission intensity. This lack of reciprocity suggests a lack of dependence on the time-dependent reduction of reductide's disulfide bond (FIGS. 1c-d).

### Distribution of Reductide During Live Cell Imaging

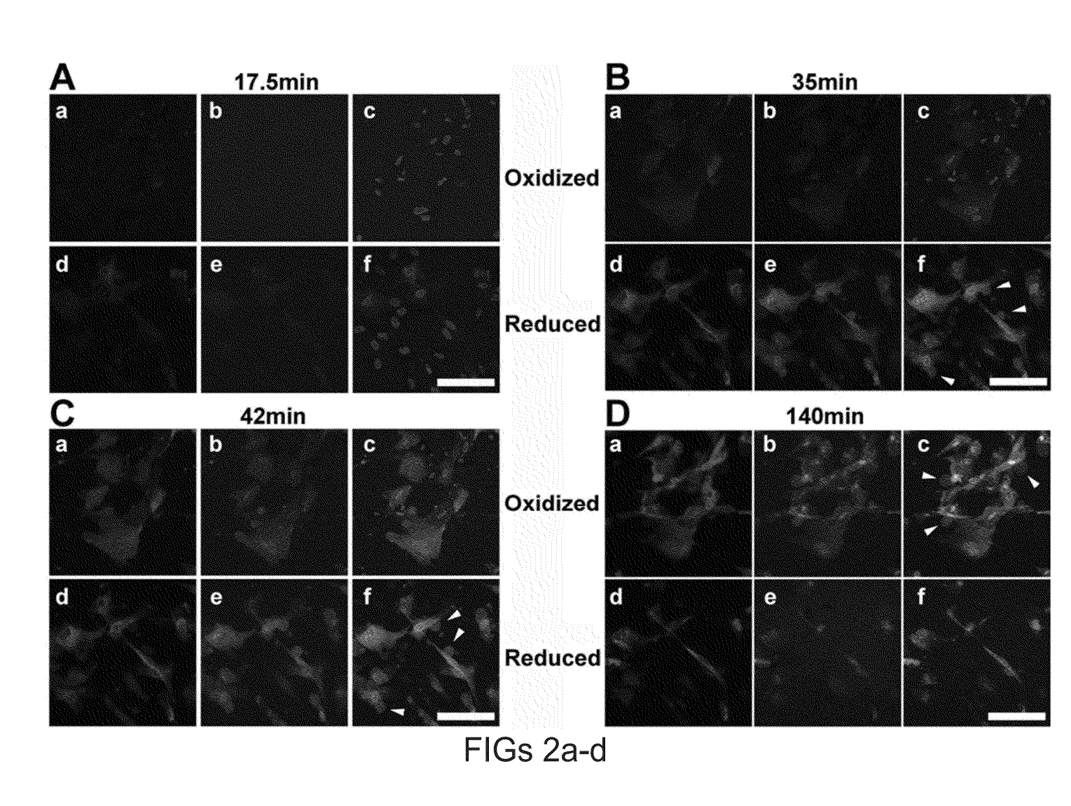

During live cell microscopy of TAMRA and FAM fluorescence, peptide uptake and cellular distribution appeared heterogeneous but essentially pan-cytosolic in BJ fibroblasts (FIGS. 2a-d). There was relative sparing of the nucleus by the TAMRA labeled cell-penetrating peptide moiety while the FAM labeled client peptide moiety appeared to distribute well within the nucleus. At later stages of reductide incubation, the FAM labeled moiety was expelled into the extracellular space via exocytic vesicles and distributed homogeneously throughout the extracellular media. The TAMRA labeled moiety was retained within cells. Both TAMRA and FAM signals appeared earlier in reduced cells (treated with NAC) than in oxidized cells (treated with CDNB), suggesting some dependence of cellular peptide uptake on cellular redox state.

Comparison of Reductide with roGFP

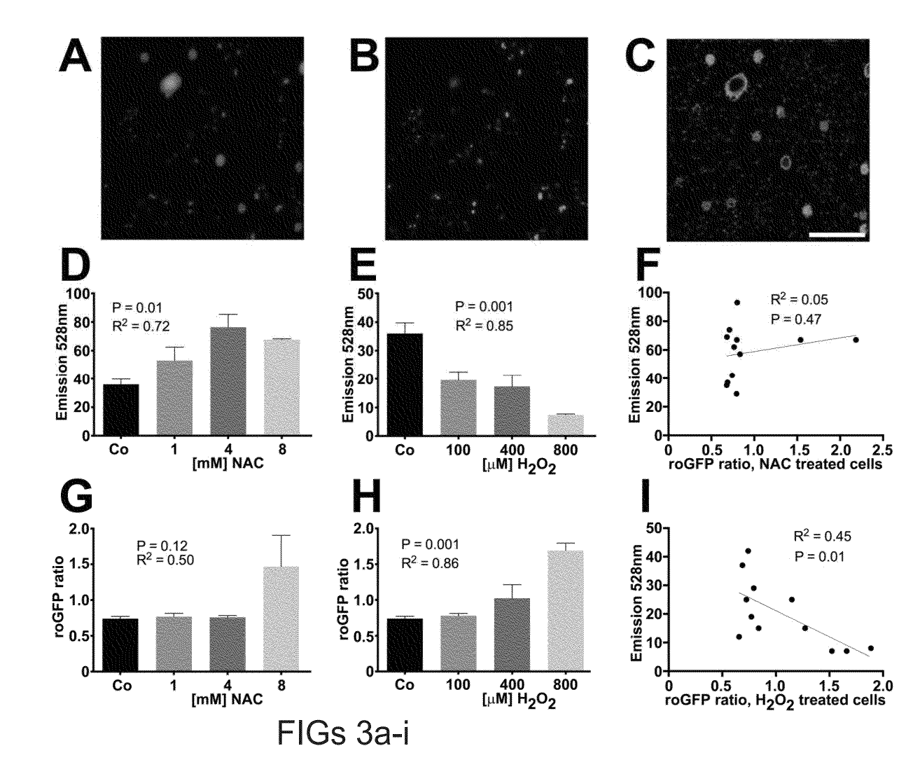

H9c2 cells were generated with stable redox sensitive green fluorescent protein (roGFP) expression that were seeded into a 96-well plate. They were pretreated with redox modifying agents (NAC or H₂O₂) followed by washing with PBS then assayed for roGFP activity using high-throughput microscopy. The microscopy assay was immediately followed by incubation with reductide in the same 96-well plate. This was followed by fluorescence plate reader assay. The ratio of roGFP emission intensities in response to excitation at 405 nm and 488 nm depends on GSSG reduction potential. The average roGFP emission ratio for each well was compared with the intensity of FAM emission for each well following incubation with reductide. There was significant correlation between roGFP emission ratio and reductide FAM signal in response to H₂O₂ treatment. There was no significant correlation between roGFP emission ratios and reductide signal following NAC pretreatment, however (FIGS. 3a-i).

Cellular Uptake and Reduction of Reductide Varies with Cellular Redox State

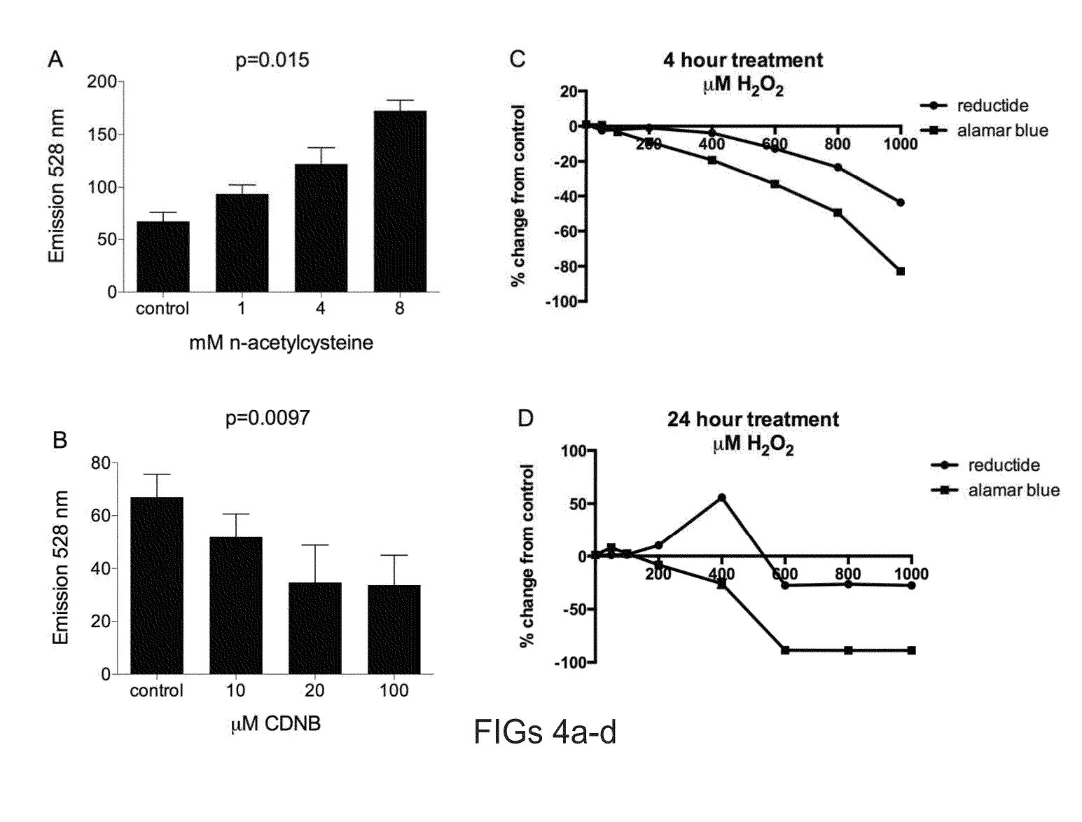

BJ fibroblasts in 96-well plates were pretreated with various redox-modifying agents followed by washing, incubation with reductide, and assessment of fluorescence by plate reader. FAM signal increased in proportion to the concentration of NAC pretreatment (FIG. 4A) and decreased in proportion to the concentration of CDNB pretreatment (FIG. 4B). Following four hours of incubation with H₂O₂, FAM signal decreased in proportion to H₂O₂ concentration used (FIG. 4C). However, following twenty-four hours of treatment with H₂O₂, FAM signal was increased for BJ fibroblasts treated with 200-400 μM H₂O₂ (FIG. 4D) but decreased with higher doses. TAMRA signal was relatively constant for each well, consistent with the idea that TAMRA is not significantly quenched by FAM and consequently not much affected by reduction of reductide's disulfide bond.

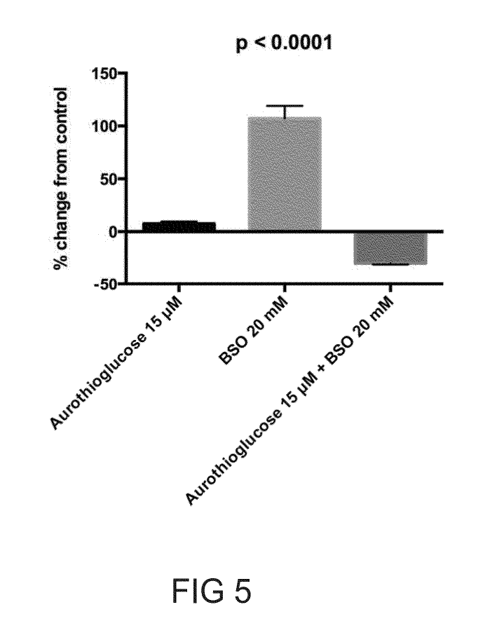

Although glutathione is the most abundant intracellular redox buffering system, the protein thioredoxin also acts as an important redox buffer. In order to test whether uptake and reduction of reductide is affected by glutathione status, thioredoxin status, or both, BJ fibroblasts were treated with the specific inhibitor of thioredoxin reductase, aurothioglucose, the inhibitor of glutathione biosynthesis, 1-buthionine sulfoximine (BSO), or both for 24 hours followed by washing, incubation with reductide for four hours, and plate reader assay. In response to treatment with aurothioglucose, there was a small increase in reductide FAM signal that was not statistically significant. There was a significant increase in signal in response to treatment with BSO and a significant decrease in signal in response to both BSO and aurothioglucose (FIG. 5), indicating that oxidative changes in both the thioredoxin and glutathione systems are required to decrease reductide signal. Thus, uptake and reduction of reductide depends on both glutathione and thioredoxin systems.

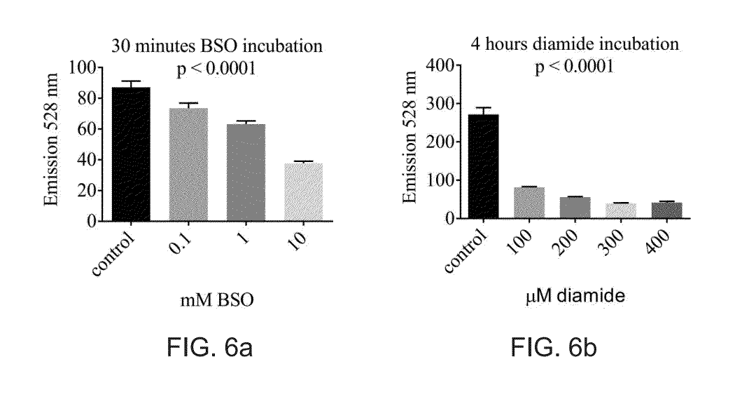

In another test to determine whether uptake and reduction of reductide is affected by glutathione status, BJ fibroblasts were treated with 1-buthionine sulfoximine (BSO), an inhibitor of glutathione biosynthesis, or diamide, a thiol oxidizing agent that decreases the cellular GSH to GSSG ratio. Treatment with either agent resulted in a significant decrease in reductide FAM signal (FIGS. 6a-b), indicating that reductide uptake and reduction are affected by cellular glutathione status. FIGS. 6a-b show that reductide uptake and reduction is inhibited by glutathione depletion. BJ fibroblasts were seeded into a 96-well plate at a density of 4,000 cells per well and allowed to attach overnight. The following day, cells were treated with the glutathione biosynthesis inhibitor 1-buthionine sulfoximine (BSO) (FIG. 6a) or the thiol oxidizing agent diamide (FIG. 6b) in normal media also containing reductide 1 μM.

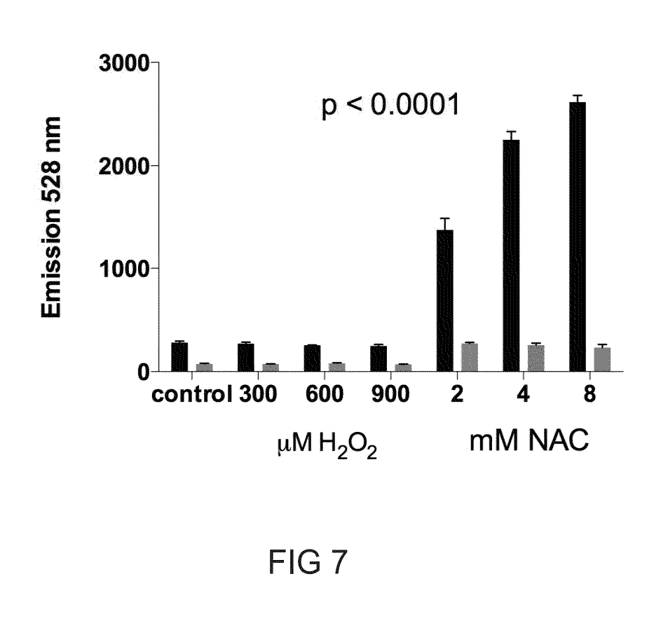

In order to test whether cellular uptake of reductide is affected by redox state, reductide fluorescence from BJ fibroblasts first incubated with reductide followed by washing and treatment with redox-modifying agents (NAC or H₂O₂) was compared with reductide fluorescence from fibroblasts first treated with redox-modifying agents and afterward incubated with reductide. FAM fluorescence was markedly decreased when cells were first incubated with reductide followed by redox-modifier treatment in comparison with cells first treated with redox-modifying agents followed by incubation with reductide (FIG. 7). FIG. 7 shows that cellular uptake of reductide is affected by redox state. To compare the effects of cellular redox modification prior to incubation with reductide with modification after incubation with reductide, BJ fibroblasts were incubated first with reductide 1 μM for one hour followed by washing with PBS and subsequent treatment with redox modifying agents (NAC or H2O2) for 60 minutes. Plate reader fluorescence results for cells treated with redox modifying agents first followed by reductide incubation (black bars) are shown in comparison with cells first incubated with reductide and afterward treated with redox modifying agents (gray bars).

Comparison with Monochlorobimane

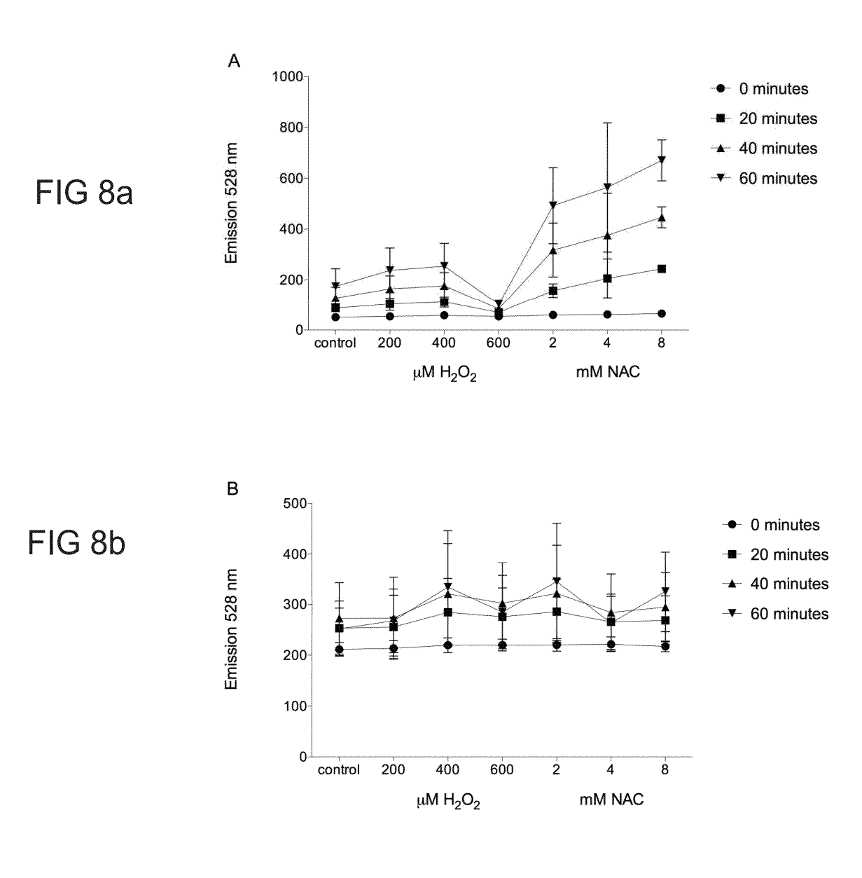

FAM signal following incubation with reductide of IMR90 cells pretreated with NAC or H₂O₂ showed dose-dependent changes in intensity. As observed in BJ fibroblasts, low doses of H₂O₂ treatment resulted in mild increases in FAM signal, while treatment with 600 μM resulted in a significant decrease in FAM signal. Signal from monochlorobimane did not show significant dependence on pretreatment dose or type of redox modifying agent (FIGS. 8a-b). FIGS. 8a-b show that a plate reader assay with reductide is more dependent on the dose of redox modifier pretreatment than the plate reader assay with monochlorobimane. Reductide (FIG. 8a) is compared with monochlorobimane (FIG. 8b), which is non-fluorescent unless conjugated to low molecular weight thiols, in IMR90 fibroblasts following pretreatment with NAC or H2O2. Cells were seeded into a 96-well plate at a density of 50,000 cells per well and allowed to attach overnight. The following day, cells were incubated with vehicle, NAC, or H2O2 at the indicated concentrations for 60 minutes followed by washing and replacement of media with assay buffer containing monochlorobimane or reductide. Reductide or monochlorobimane signal was ascertained at the indicated time points of incubation.

### Flow Cytometry

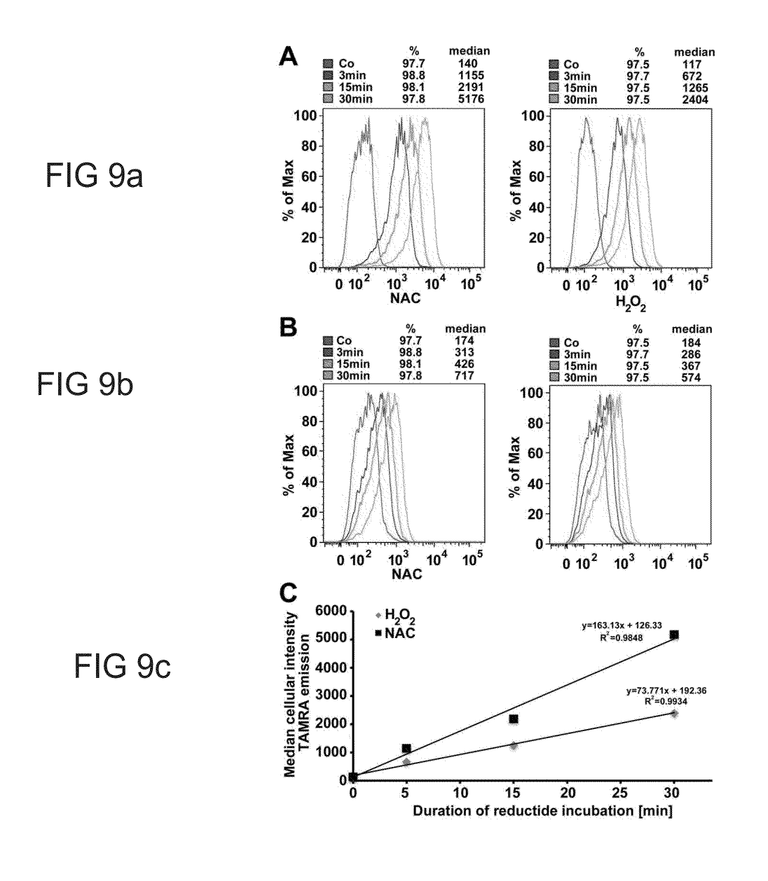

IMR90 fibroblasts incubated with reductide for various time periods exhibited a time-dependent increase in both TAMRA and FAM signals as detected by flow cytometry. TAMRA signal was strongest in cells pretreated with NAC 4 mM. FAM signal was relatively weaker and exhibited less temporal resolution than TAMRA (FIGS. 9a-c). This is consistent with cellular exportation of FAM-labeled CLKANL, which was observed during live cell microscopy. The time-dependent increase in TAMRA signal is attributable to continuous uptake of reductide over time. No increase in nonviable cells as observed by side-scatter or DAPI signal was seen in cells incubated with reductide vs. controls or in cells pretreated with H₂O₂. FIGS. 9a-c provide flow cytometry data showing time dependent increases in cellular TAMRA (9a) and FAM (9b) signals in response to incubation with reductide 1 μM for 3, 15 or 30 minutes. IMR90 cells were seeded into 10 cm dishes at a density of 1.8×106 and allowed to attach overnight. The following day, cells were pretreated with NAC 4 mM or H2O2 600 μM for 60 minutes prior to peptide incubation. Median cellular TAMRA emission intensity as a function of time is shown (9c).

### Reductide Response to a Small Library of Redox Modifying Compounds

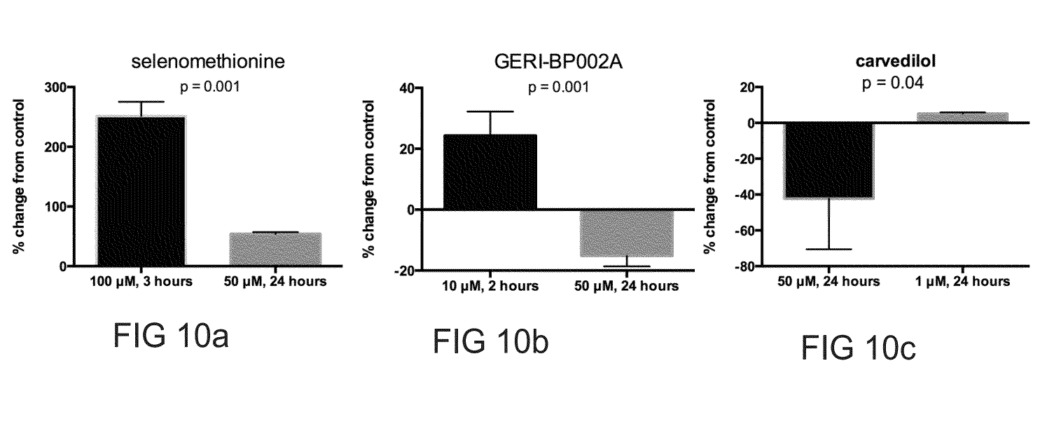

BJ fibroblasts were seeded into a 96-well plate at a density of 4,000 cells per well and allowed to attach overnight. The following day, cells were incubated in normal cell media supplemented with 50 μM of a redox-modifying compound from the redox library distributed from Enzo Life Sciences. Each redox compound was used to treat three wells for 24 hours. Afterward, cells were washed with PBS followed by incubation in reductide 1 μM dissolved in normal media for four hours. FAM signal from reduction of reductide's disulfide bond was assayed in a plate reader. Most compounds in the library are classified as antioxidants. FAM signal was significantly increased in cells treated with 65 of the compounds or 77.4% of the library, and significantly decreased in response to treatment with nine compounds or 10.7% of the library. The remaining compounds did not result in a statistically significant change in FAM signal compared to vehicle treated cells. It should be noted that the screening conditions (50 μM concentration, 24 hour drug incubation) were not optimized for each drug individually. That many antioxidants can act as pro-oxidants if their concentration is sufficiently high is well known. Some antioxidative compounds such as GERI-BP002A and carvedilol resulted in a significant decrease in FAM signal following incubation at 50 μM for 24 hours. When retested at new concentrations, different results were obtained (FIG. 10a-c), showing an increase in signal expected for reduction. FIGS. 10a-c show that antioxidative compounds GERI-BP002A and carvedilol have pleiotropic effects on redox state depending on concentration. At concentrations of 50 μM, these compounds caused apparent oxidation as indicated by a decrease in FAM signal relative to vehicle treated cells. At lower concentrations, there was an increase in FAM signal consistent with reduction. By comparison, selenomethionine, an augmenter of thioredoxin reductase and glutathione peroxidase, caused a significant increase in FAM signal consistent with reduction at all concentrations tested. Other representative results following treatment at 50 μM for 24 hours are shown in Table 1.

TABLE 1

| | | % change | P value |
|---|---|---|---|
| | | from | comparison |
| Compound name | Class | control | with control |
| Ethoxyquin | Nonphenolic antioxidant | 155.4 | 9.5E−05 |
| Seratrodast | Quinone antioxidant | 134.9 | 9.1E−05 |
| Retinyl palmitate | Radical scavenger | 123.5 | 2.5E−03 |
| Idebenone | Quinone antioxidant | 120.8 | 3.3E−02 |
| β-carotene | Radical scavenger | 111.1 | 3.8E−04 |
| Ebselen | GSH peroxidase mimetic | 77.2 | 0.00023 |
| Cumene | Aryl hydroperoxide | −30.8 | 1.7E−03 |
| hydroperoxide | | | |
| N-Ethylmaleimide | Thiol trap | −71.7 | 0.00001 |

In Table 1, BJ fibroblasts were seeded into a 96-well plate at a density of 4,000 cells per well and allowed to attach overnight in preparation for incubation with 50 μM of redox modifying compounds dissolved in cell media for 24 hours. Cells were subsequently washed with PBS and incubated with reductide 1.5 μM dissolved in cell media for four hours. FAM signal was assayed in a plate reader. The redox modifying compounds were obtained as an 84 compound library from Enzo Life Sciences. Percentage change in reductide signal in comparison with vehicle treated cells are shown for a subset of the redox modifying compounds.

### Use of Reductide to Discover Novel Antioxidative Compounds

Reductide was used to discover compounds with novel antioxidative activity by screening three compound collections: 1) a collection of 480 novel compounds from the Chemistry Department at the University of Utah; 2) the 2000 compound SPECTRUM library, which includes many known drugs; and 3) a collection of 400 natural products assembled by researchers at the University of Utah.

Cells were seeded into 96-well plates and allowed to attach overnight. The following day, cell media was exchanged for media containing compounds from chemical libraries (4 micromolar, 1% DMSO). Each 96-well plate had negative replicate wells containing 1% DMSO in cell media. Cells were incubated for 24-hours with chemical library members. Following incubation, cells were washed one time with PBS, and reductide (1 μM) in cell media was added to each well, after which cells were incubated for 3-4 hours. Reductide signal was then assayed in plate reader.

A “hit” was defined as a chemical compound that resulted in reductide signal intensity more than 5 standard deviations above the mean signal for cells pretreated with only 1% DMSO. Hits were confirmed by repeat testing in triplicate with reductide.

A secondary test was performed to evaluate the ability of each hit to protect cells from oxidative stress. Cells in 96-well plates were incubated with hits dissolved to 4 μM in cell media for 24 hours (three wells per compound). Cells were then washed once with PBS, and treated with hydrogen peroxide dissolved in cell media for four hours. Cells were then washed once with PBS, and cell viability was assayed with Alamar Blue.

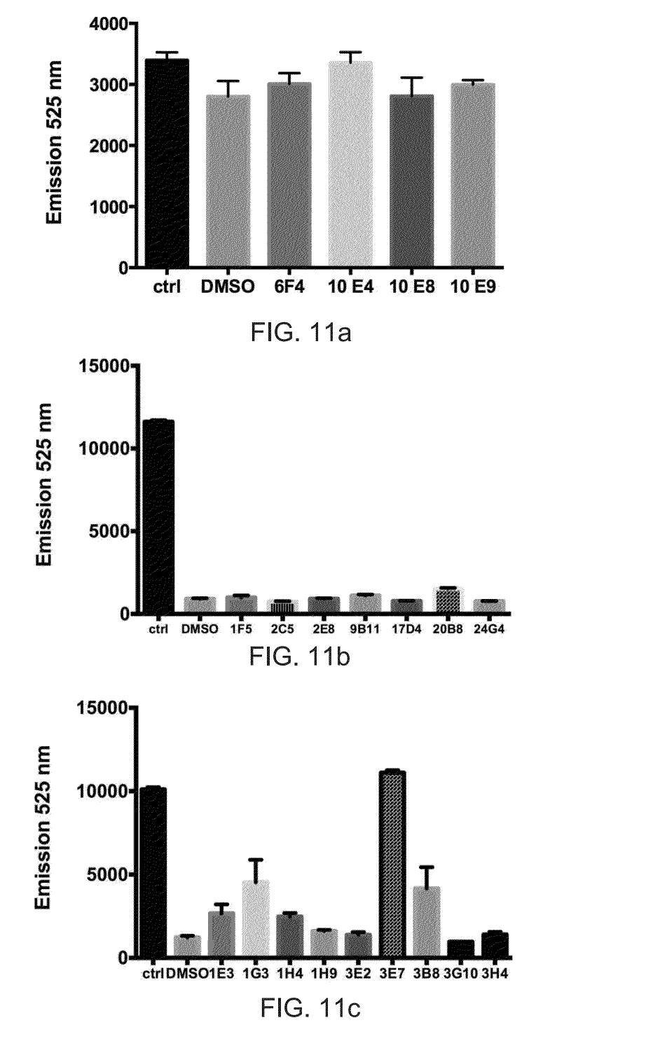

FIGS. 11a-c shows results from the above described testing. FIG. 11a shows the viability of BJ fibroblasts 2 hours following treatment with 240 μM hydrogen peroxide. Control cells were not treated with hydrogen peroxide. Every other category, including DMSO, was treated with 240 μM hydrogen peroxide for four hours prior to viability assessment with Alamar Blue. P-value for 10E4 vs. DMSO is 0.04, p-value for 10E4 vs. control is 0.98, and p-value for DMSO vs. control is 0.02. FIG. 11b shows the viability of H9C2 cells 24 hours after treatment with 600 μM hydrogen peroxide. 9B11 (thiram) and 20B8 (anthothecol) signals are significantly higher than DMSO (p-values=0.007 and 0.00002, respectively). FIG. 11c shows the viability of BJ fibroblasts 24 hours following treatment with 980 μM hydrogen peroxide. Control cells were not treated with hydrogen peroxide. DMSO pre-treated cells and every other category was treated with 980 μM hydrogen peroxide for four hours prior to the addition of Alamar Blue. P-values for 1G3 (3-bromohomofascaplysin), 3E7 (penicillic acid), and 3B8 (epicorazine A) vs. DMSO are 0.01, <0.00001, and 0.02, respectively. 3E7 viability is significantly higher than control (p-value=0.001).

### Discussion

### Reductide Uptake as Well as Reduction Depends on Cellular Redox State

The rate of development of FAM fluorescence following incubation of cells with reductide depends broadly on at least two composite steps: 1) cellular uptake and internalization of reductide and 2) reduction of reductide's disulfide bond. If differences in redox state only affected the rate of step 2, it is unlikely that reductide signal could be used to distinguish intracellular redox state in most living cells. This assertion is based upon the fact that the rate of development of FAM signal during incubation of reductide in TBS buffer containing various ratios of GSH/GSSG is not significantly different between 2 mM GSH/1.5 mM GSSG (GSSG reduction potential −164 mV at 25° C., using the Nernst equation) and 5 mM GSH (GSSG reduction potential less than −200 mV). These values nearly span the range of GSSG reduction potentials for viable cells. Variation in the rate of step 2 is therefore likely small throughout the range of intracellular reduction potentials in living cells. Consequently, intracellular reduction potential must affect step 1 if development of FAM signal is significantly different between cells with different redox states. Indeed, two of our experiments suggest that it does: 1) TAMRA signal, which does not require reduction of reductide's disulfide bond for detection, occurs earlier by fluorescence microscopy in reduced cells than in oxidized cells incubated with reductide; 2) development of FAM signal in a plate reader assay is attenuated and there is a smaller difference in signal between cells treated with reducing or oxidizing agents when incubation with reductide precedes treatment with redox-modifying agents. In this latter experiment, redox-dependent differences in rates of cellular uptake and internalization of reductide are controlled for by not modifying redox state until after reductide has been internalized. Redox dependent differences in development of FAM signal are much larger when incubation with reductide follows treatment with redox-modifying agents, suggesting that cellular uptake and internalization is an important step in redox-dependent development of FAM signal. This may partially explain why 2-deoxyglucose, an inhibitor of glucose-6-phosphate dehydrogenase and pro-oxidant, inhibits cellular uptake of MAP. This property of MAP uptake offers potential for redox-dependent, targeted delivery of drugs or imaging agents using MAP-like constructs.

### Pro-Oxidants Activate an Antioxidative Response

Pretreatment of human fibroblasts with lower doses of H₂O₂ (200-400 μM) resulted in increased FAM fluorescence, indicating an increase in cellular reduction. In contrast, treatment with 600 μM or higher doses of H₂O₂ was associated with a decrease in FAM fluorescence. This finding may be explained by the fact that low dose H₂O₂ stimulates an antioxidative, and hence reductive, response that is overcome by higher doses of H₂O₂. A number of published investigations support the plausibility of this idea. For example, low and moderate doses of H₂O₂ in pulmonary endothelial cells caused nuclear accumulation of the redox-sensitive Nrf2 transcription factor and increased antioxidant response element (ARE)-dependent gene expression; in contrast, there was down-regulation of ARE-mediated gene expression and nuclear exclusion of Nrf2 at high dose H₂O₂ in the same cells. Similarly, treatment of human umbilical vein endothelial cells with low dose H₂O₂ caused upregulation of thioredoxin-1 and inhibition of apoptosis after serum deprivation, whereas treatment with higher dose H₂O₂ resulted in no change in thioredoxin-1 expression but increased susceptibility to apoptosis. Jarrett and Boulton reported that exposure of retinal pigment epithelial cells to sublethal doses of H₂O₂ caused upregulation of catalase, glutaperoxidase, Cu/Zn superoxide dismutase, and resistance to death caused by high dose H₂O₂. In V79 fibroblasts, exposure to low dose H₂O₂ caused upregulation of catalase by improving stability of its mRNA. This was mediated by activation of p38 mitogen-activated kinase. In another report, exposure of V79 cells to low dose H₂O₂ resulted in increased GSH content, increased activity of Cu/Zn superoxide dismutase, catalase, and glutaperoxidase, and increased resistance to cell killing by H₂O₂ and cisplatin. The oxidant dose range that is most likely to stimulate an overall antioxidative response is likely to vary by cell type and species.

It is recognized that intracellular redox state remains dynamic and highly dependent on degree of cellular differentiation, density, and proliferative potential. Variations in redox state are linked to cell cycle progression. Redox signaling plays a role in the pathogenesis of cardiomyopathy, cardiovascular disease, neurodegenerative disorders, and cancer, to name a few. Redox changes modulate apoptosis; depletion of reduced glutathione or moderate oxidative changes induce apoptosis, while more severe oxidation inhibits apoptosis, probably through oxidation of caspases, resulting in cell death by necrosis. Redox-based delivery of pharmaceuticals thus has the potential to modify a variety of disease processes.

As such, cellular uptake and reduction of model amphipathic peptide conjugated through disulfide linkage to a signal cargo varies by cellular redox state and can be used to interrogate relative redox changes in cells.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A conjugate for detecting cellular uptake and cellular redox state, comprising:
a first segment including a cell penetrating peptide conjugated to a first detection molecule; and
a second segment including a cargo peptide conjugated to a second detection molecule, wherein the first segment and the second segment are coupled together by a redox-sensitive linkage, and wherein the first detection molecule and the second detection molecule have properties that allow linked proximity detection.

2. The conjugate of claim 1, wherein the first detection molecule and the second detection molecule include a fluorophore/quencher pair.

3. The conjugate of claim 2, wherein the first detection molecule is 5(6)carboxytetramethylrhodamine-cysteine and the second detection molecule is fluorescein amidite, wherein the 5(6)carboxytetramethylrhodamine-cysteine quenches fluorescence of fluorescein amidite when in linked proximity.

4. The conjugate of claim 3, wherein the cell penetrating peptide is conjugated at an N-terminus to the 5(6)carboxytetramethylrhodamine-cysteine.

5. The conjugate of claim 3, wherein the cargo peptide is conjugated at an N-terminus to the fluorescein amidite.

6. The conjugate of claim 1, wherein the cell penetrating peptide is a cationic cell penetrating peptide.

7. The conjugate of claim 1, wherein the cell penetrating peptide includes a member selected from the group consisting of Tat-derived cell penetrating peptides, penetratins, transportan and transportan-related peptides, model amphipathic peptides, and combinations thereof.

8. The conjugate of claim 1, wherein the redox sensitive linkage includes a member selected from the group consisting of disulfide linkages, substrates for enzymes controlled by redox state, substrates for enzymes which are up regulated or expressed in response to changes in cellular redox state, and combinations thereof.

9. The conjugate of claim 1, wherein the redox sensitive linkage is a disulfide linkage.

10. The conjugate of claim 1, wherein the cargo peptide is from about one to about fifty amino acids in length.

11. The conjugate of claim 1, wherein the cargo peptide can include a member selected from the group consisting of fluorescent proteins, bioluminescent proteins, and combinations thereof.

12. The conjugate of claim 1, wherein the cargo peptide is CLKANL (SEQ ID 001).

13. A method of detecting cellular redox state, comprising
introducing the conjugate of claim 1 into a cell; and
measuring linked proximity of the first detection molecule and the second detection molecule to detect cleavage of the redox sensitive linkage to determine a cellular redox state.

14. The method of claim 13, further including detecting at least one of the first or second detection molecules to determine uptake of the conjugate by the cell.

15. The method of claim 14, wherein determining uptake of the conjugate occurs prior to detectable cleavage of the redox sensitive linkage.

16. The method of claim 13, wherein the cell is a population of cells.

17. The method of claim 16, wherein the cellular redox state is monitored across the population of cells to determine a relative change in cellular redox state.

18. A kit for detecting cellular redox state, comprising:
a housing containing:
the conjugate of claim 1 in a biologically suitable carrier;
at least one reagent for use with the conjugate in detecting cellular redox state; and
instruction materials describing utilization of the conjugate and the at least one reagent to detect the cellular redox state.
